(19) United States
(12) Patent Application Publication
(10) Pub. No.: US 2018/0177488 A1
(43) Pub. Date: Jun. 28, 2018

(54) FLUID FILLED IMAGING CATHETER

(71) Applicant: ACIST Medical Systems, Inc., Eden Prairie, MN (US)

(72) Inventors: Reena Pathania, Fremont, CA (US); Laihlani Montoya, San Jose, CA (US)

(21) Appl. No.: 15/388,325

(22) Filed: Dec. 22, 2016

(51) Int. Cl. A61B 8/00; A61B 8/12; A61B 8/08

(52) U.S. Cl. CPC A61B 8/4281; A61B 8/12; A61B 2562/12; A61B 8/4422; A61B 8/085

(57) ABSTRACT

Imaging catheter assemblies as well as related methods of manufacturing and imaging are disclosed. The imaging catheter assembly can include an interior lumen containing an intravascular imaging device as well as an acoustic fluid medium comprising ethanol. Such an imaging catheter assembly can be contained within a package, such that when the imaging catheter assembly is removed from the package it can begin generating image data without needing to perform one or more preparation steps on the imaging catheter assembly. Related methods of manufacturing include applying a vacuum to an interior lumen of an imaging catheter assembly and filling the interior lumen with an acoustic fluid medium comprising ethanol. After filling the interior lumen, the imaging catheter assembly is packaged within a packaging container for later use.

### TECHNICAL FIELD

This disclosure relates generally to imaging catheter assemblies and related methods, such as methods of imaging employing an imaging catheter and methods of manufacturing an imaging catheter.

### BACKGROUND

Medical imaging techniques generally can be used to collect data and generate in-vivo visualization of anatomical areas of interest. One such example is intravascular imaging, where vascular structures and lumens may be imaged. For instance, intravascular imaging may be used to produce one or more images of the coronary artery lumen, coronary artery wall morphology, and devices, such as stents, at or near the coronary artery wall. Images generated using medical imaging techniques can be useful for diagnostic purposes, such as identifying diagnostically significant characteristics of a vessel.

To collect image data, intravascular imaging procedures generally use an imaging probe positioned within a catheter that is inserted within a vascular structure. However, before image data can be collected steps are usually taken to prepare the catheter for use. For example, an interior of the catheter may be flushed with a flushing solution. In instances where the imaging probe is an ultrasonic imaging probe, this flushing may continue to take place during imaging. In addition to increasing user burden and time needed to collect image data, preparing the catheter for use can necessitate additional accessories, such as syringes, tubing extension sets, and flushing solution.

### SUMMARY

This disclosure in general provides embodiments relating to a fluid filled imaging catheter that can begin collecting imaging data (e.g., immediately) upon connecting the imaging catheter to an imaging engine. As one example, a catheter housing can define an interior lumen that is filled with an acoustic fluid medium including ethanol and/or water (e.g., sterilized). This acoustic fluid medium may surround an ultrasound transducer positioned within a portion of the lumen. The described catheter housing can be packaged for a prolonged period of time (e.g., months) without detrimentally affecting properties of the ultrasound transducer. In addition, when the catheter housing is removed from the packaging at a later time, the noted acoustic fluid medium can serve to transmit ultrasound energy between the transducer and the surrounding vessel in a manner that can facilitate collection of quality image data.

Embodiments of the fluid filled imaging catheter may not require traditional preparation steps, such as fluid flushing through and out of the catheter, prior to collecting image data. Likewise, embodiments of the fluid filled catheter may eliminate the need for certain accessories (e.g., syringes, tubing extension sets, and flushing solution) used for such traditional catheter preparation steps. As a result, embodiments disclosed herein can provide a variety of useful advantages, including a reduction in user burden and time needed to collect image data. Such advantages may be particularly useful in intravascular imaging applications, where it can be desirable to reduce the time during which a patient is catheterized and the number of accessories required in a sterile environment. Furthermore, techniques used to pre-fill the imaging catheter assembly with the acoustic fluid medium (e.g., application of a vacuum to an interior lumen to induce capillary action) may prevent air bubble formation within the imaging catheter assembly. This is an issue that can degrade image data quality when traditional preparation steps and accessories are utilized to introduce fluid into the imaging catheter assembly.

One exemplary embodiment includes an imaging catheter assembly. The imaging catheter assembly has a housing enclosed within a package. The housing defines an interior lumen of the imaging catheter assembly and an ultrasound transducer is disposed within the interior lumen. An acoustic fluid medium including ethanol is contained within the interior lumen. In a further exemplary embodiment of an imaging catheter assembly, the housing defines a proximal portion and a distal portion, with the distal portion being an enclosed space and having a guide wire receiving component.

Another exemplary embodiment includes a method of manufacturing an imaging catheter. The method includes applying a vacuum device to an interior lumen that is defined by a catheter housing having an ultrasound transducer disposed within the interior lumen. The method further includes filling the interior lumen with an acoustic fluid medium (e.g., a degassed acoustic fluid medium) that includes ethanol. In addition, the method includes packaging the catheter housing within a packaging container for later use after filling the interior lumen with the acoustic fluid medium.

A further exemplary embodiment includes a method of imaging. The method includes removing packaging enclosing an imaging catheter. The imaging catheter removed from the packaging includes a catheter housing defining an interior lumen, an ultrasound transducer disposed within the interior lumen, and an acoustic fluid medium including ethanol within the interior lumen. The method also includes, after removing the packaging, connecting the imaging catheter to an imaging engine and delivering the imaging catheter over a guide wire to a region of interest within a vessel. The method also includes emitting and receiving ultrasound energy between the ultrasound transducer, the acoustic fluid medium within the interior lumen, and vessel at the region of interest. Additionally, the method includes conveying image data from the ultrasound transducer to the imaging engine. This image data corresponds to the reflected ultrasound energy received at the ultrasound transducer.

The details of one or more examples are set forth in the accompanying drawings and the description below. Other features, objects, and advantages will be apparent from the description and drawings, and from the claims.

### BRIEF DESCRIPTION OF DRAWINGS

The following drawings are illustrative of particular examples of the present invention and therefore do not limit the scope of the invention. The drawings are not to scale (unless so stated) and are intended for use in conjunction with the explanations in the following detailed description. Examples of the present invention will hereinafter be described in conjunction with the appended drawings.

FIG. 1 is an illustrative example of a system configured to perform intravascular imaging.

FIG. 2 is a front view of an embodiment of an imaging catheter assembly including data vectors propagated by a transducer of the imaging catheter assembly.

FIG. 3A is a side elevational view of an embodiment of an imaging catheter assembly.

FIG. 3B is a longitudinal cross-sectional view of the imaging catheter assembly of FIG. 3A.

FIG. 4A is a perspective view of a distal portion of the imaging catheter assembly of FIG. 3A

FIG. 4B is a cutaway perspective view of the distal portion shown in FIG. 4A.

FIG. 5 is a flow diagram illustrating an exemplary method of manufacturing an imaging catheter assembly.

FIG. 6 is a flow diagram illustrating an exemplary method of imaging using an imaging catheter assembly.

### DETAILED DESCRIPTION

The following detailed description is exemplary in nature and is not intended to limit the scope, applicability, or configuration of the invention in any way. Rather, the following description provides some practical illustrations for implementing examples of the present invention. Those skilled in the art will recognize that many of the noted examples have a variety of suitable alternatives.

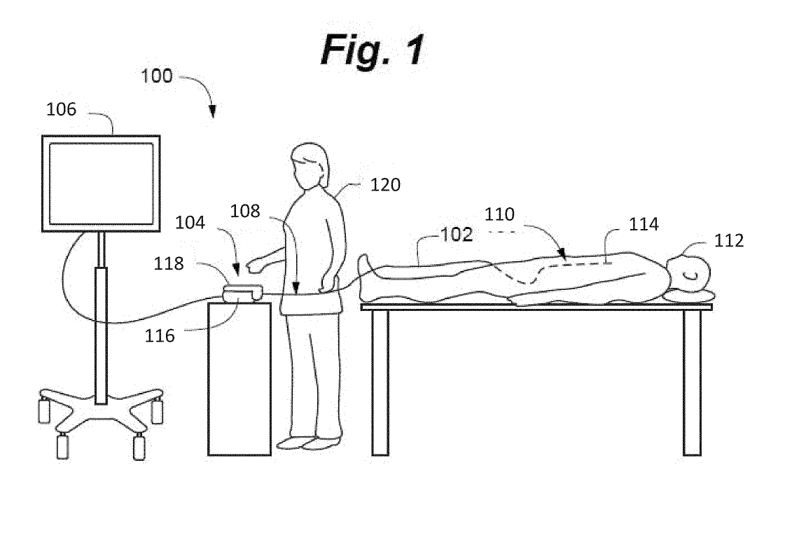

FIG. 1 illustrates an example of a system 100 that may be configured to perform intravascular imaging. System 100 can include an imaging catheter assembly 102, a translation device 104, and an imaging engine 106. The imaging catheter assembly 102 may include a proximal end 108 and a distal end 110 configured to be inserted into a vessel of a patient 112. In one example, imaging catheter assembly 102 may be inserted into the patient 112 via the femoral artery and guided to an area of interest within the patient 112. The broken lines in FIG. 1 represent portions of imaging catheter assembly 102 within the patient 112.

In some examples, the imaging catheter assembly 102 can include an intravascular imaging device 114 configured to generate image data. Intravascular imaging device 114 can be in communication with imaging engine 106. In some embodiments, intravascular imaging device 114 is an ultrasound transducer configured to emit and receive ultrasound energy and generate ultrasound imaging data. The image data generated by the imaging device 114 can represent a cross-section of an area of interest within the patient 112 at the location of the imaging device 114. The image data generally will represent a plurality of image items at the cross-sectional location of the imaging device 114, such as, for example, various layers of a vessel of the patient 112 and/or any accumulated matter within the vessel (e.g., plaque).

The translation device 104 can be configured to translate intravascular imaging device 114 of imaging catheter assembly 102. The translation device 104 may comprise a linear translation system (LTS) 116. The LTS 116 may be mechanically engaged with imaging catheter assembly 102 and configured to translate imaging catheter assembly 102 a controlled distance within the patient 112 during a translation operation, for example a pullback or push-forward operation. The system 100 may comprise a patient interface module (PIM) 118 configured to interface the translation device 104 with the catheter assembly 102. Translating the imaging device 114 can allow for cross-sectional image data to be collected at various longitudinal locations within a vessel of the patient 112. This cross-sectional image data at various longitudinal locations can then be compiled, in some applications, to generate a longitudinal cross-sectional image of an area of interest.

The imaging engine 106 can be in communication with intravascular imaging device 114 and/or translation device 104. According to some examples, the imaging engine 106 may comprise at least one programmable processor. In some examples, the imaging engine 106 may comprise a computing machine including one or more processors configured to receive commands from a system user 120 and/or display data acquired from imaging catheter assembly 102 via a user interface thereof. The computing machine may include computer peripherals (e.g., keyboard, mouse, electronic display) to receive inputs from the system user 120 and output system information and/or signals received from imaging catheter assembly 102 (e.g., rendered images). In some examples, the user interface of the computing machine may be a touchscreen display configured to act as both an input device and an output device. In some examples, imaging engine 106 may include memory modules for storing instructions, or software, executable by the one or more processors.

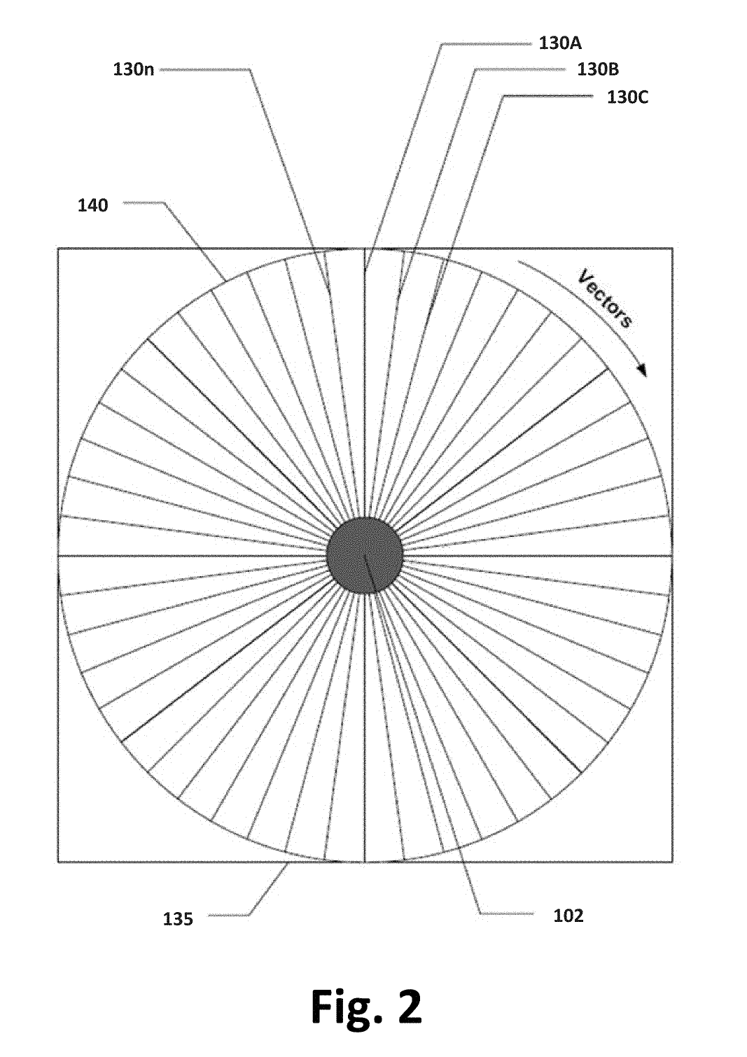

FIG. 2 illustrates a schematic front view of the imaging catheter assembly 102 used with the intravascular imaging system previously described. More specifically, FIG. 2 illustrates a front view of the distal end of the imaging catheter assembly 102 where the imaging device is located. FIG. 2 also shows data vectors propagated by the imaging device, for instance an ultrasound transducer, to generate image data when positioned with a vessel of the patient. As noted, the imaging device can be in communication with the imaging engine and communicate the image data to the imaging engine.

In the example of FIG. 2, the imaging catheter assembly 102 may be configured to rotate the imaging device relative to an outer housing (e.g., a sheath) of the imaging catheter assembly 102. Where the imaging device is an ultrasound transducer, the ultrasound transducer may be configured to generate ultrasound data by emitting and receiving ultrasound energy. Ultrasound data vectors are illustrated in FIG. 2 as indicative of ultrasound energy emitted and received by the ultrasound transducer at different rotational positions. More specifically, each data vector is representative of ultrasound data collected by the ultrasound transducer at different rotational positions of the ultrasound transducer within the imaging catheter assembly 102 housing. A number of the data vectors (e.g., each data vector) can, in some embodiments, be acquired at different times.

As shown in FIG. 2, the ultrasound transducer of imaging catheter assembly 102 may generate ultrasound data on a vector-by-vector basis as the transducer is rotated. For example, the ultrasound transducer may initially acquire an ultrasound data vector 130A and continue to acquire vectors 130B, 130C through 130n as the ultrasound transducer is rotated clockwise. Accordingly, vectors 130A-130n can be representative of a full 360 degree rotation of the ultrasound transducer within a vessel and make up a single frame of image data. The number of data vectors acquired per rotation may vary depending on the application of the imaging catheter assembly 102. For instance, in some embodiments, the imaging catheter assembly is configured to generate between about 500 and about 5000 vectors per rotation. For example, in an embodiment generating 512 vectors per rotation (e.g., frame) the angle between data vectors may then be characterized as approximately 2π/512 radians or 360/512 degrees. In an example of an imaging catheter assembly configured to generate 4096 vectors per rotation (e.g., frame), the angle between data vectors may be approximately 2π/4096 radians or 360/4096 degrees. FIG. 2 also provides a representation of a data frame 135 that comprises emitted and received vectors 130A-130n. A field of view 140 of the imaging catheter assembly 102 may be based on the magnitude of the data vectors propagated by the ultrasound transducer and may vary to suit a specific application. The magnitude of the data vectors may be based on a number of factors, for example, the frequency of the emitted wave (e.g., 40 MHz, 60 MHz) and/or the power level of the wave. In some embodiments, the ultrasound transducer of imaging catheter assembly 102 can emit acoustic energy at differing frequencies within the single frame 135.

Having described an exemplary intravascular imaging system and the generation of image data, this disclosure will now describe details related to embodiments of the imaging catheter assembly used in such system to generate image data.

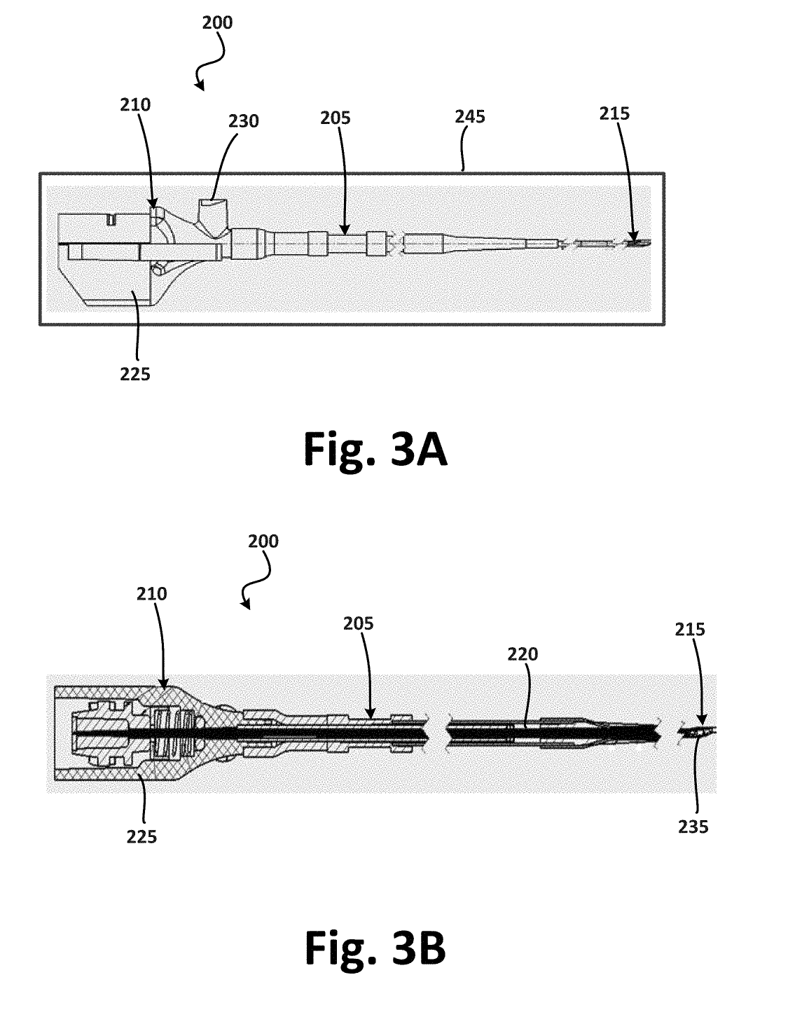

FIGS. 3A and 3B illustrate an exemplary embodiment of an imaging catheter assembly 200. In particular, FIG. 3A shows a side elevational view of the imaging catheter assembly 200 and FIG. 3B shows a longitudinal cross-sectional view of the imaging catheter assembly 200 of FIG. 3A.

The illustrated imaging catheter assembly 200 includes a housing 205. In some embodiments, a portion, or an entirety, of the housing 205 can be formed by a catheter sheath. The housing 205 can define a proximal portion 210 generally at or near one end and a distal portion 215 generally at or near an opposite longitudinal end of the housing 205. When the imaging catheter assembly 200 is in use, such as shown in FIGS. 1 and 2, the proximal portion 210 can generally be positioned outside of a patient while the distal portion 215 can generally be positioned inside of the patient (e.g., within a vessel of the patient). The housing 205 may also define an interior lumen 220 of the imaging catheter assembly 200. As shown in FIG. 3B, the interior lumen 220 may, in some embodiments, extend from the proximal portion 210 to the distal portion 215. As will be described further below, the interior lumen 220 can contain an acoustic fluid medium. In one example, a volume of the interior lumen 220 which extends from the proximal portion 210 to the distal portion 215 is substantially filled with the acoustic fluid medium.

The proximal portion 210 can include a catheter hub 225. The hub 225 may, in some instances, define a port 230. The port 230 can be in fluid communication with the interior lumen 220 and, accordingly, located at any location on the hub 225 where it is in fluid communication with the interior lumen 220. The port 230 may be adapted to receive a vacuum device. As such, the port 230 can include one or more structures configured to couple (e.g., in a fluid tight manner) to the vacuum device. In one example, the port 230 can take the form of a luer connection component, for instance where the port 230 is a female luer component and the vacuum device is a male luer component. In another example, the port 230 can be threaded to receive corresponding threading of the vacuum device. Because the port 230 is in fluid communication with the interior lumen 220, coupling the vacuum device to the port 230 brings the vacuum device into fluid communication with the interior lumen 220. In some instances, the interior lumen 220 can be filled with the acoustic fluid medium using the vacuum device. In one such instance, the acoustic fluid medium can be introduced into the interior lumen 220 through the same port 230 to which the vacuum device is coupled. This may include introducing the acoustic fluid medium into the interior lumen 220 through the vacuum device itself, and can include degassing the acoustic fluid medium. In this instance, the port 230 may be the only opening of the imaging catheter assembly 200. In another such instance, the acoustic fluid can be introduced into the interior lumen 220 through a separate fluid opening in the catheter hub 225 when present.

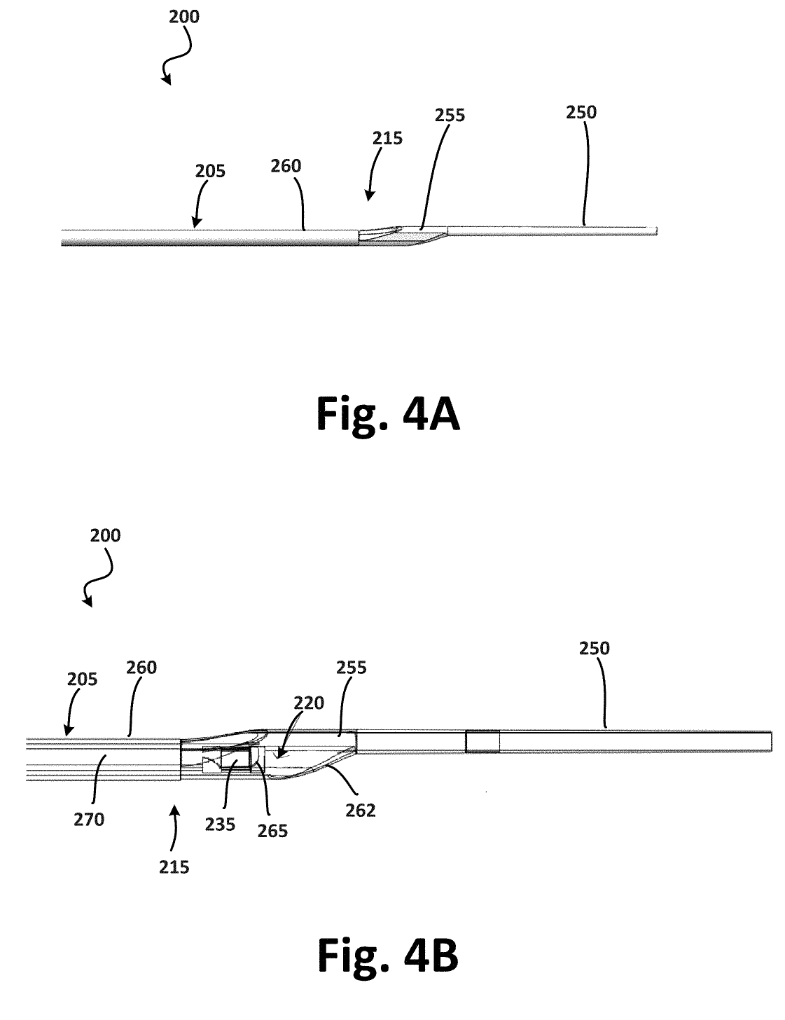

The distal portion 215 can include an intravascular imaging device 235. In the illustrated example, the intravascular imaging device 235 is an ultrasound transducer. The ultrasound transducer can be positioned within the interior lumen 220, such as at a distal portion of the interior lumen 220 as shown. As previously described, the ultrasound transducer can be coupled to a drive cable (best seen in FIG. 4B) also located within the interior lumen 220. The distal portion 215 may be adapted to receive a guide wire to guide the distal portion 215 to a region of interest within the vessel of the patient and/or translate the distal portion 215 within the vessel, such as during image data generation. In other embodiments, the distal portion 215 may be directly guided into the vessel without the use of the guide wire.

As shown in FIG. 3A, the imaging catheter assembly 200 is enclosed within a packaging container 245. The packaging container 245 may seal the imaging catheter assembly 200 from an ambient environment and thereby maintain the imaging catheter assembly 200 in a sterile state. In the illustrated example, the imaging catheter assembly 200 can be fully assembled when enclosed within the packaging container 245. For instance, enclosed within the packaging container 245 can be the housing 205 including the interior lumen 220 containing an acoustic fluid medium and the ultrasound transducer. In this way, once the imaging catheter assembly 200 is removed from the packaging container 245 the imaging catheter assembly 200 simply need be connected to the imaging engine and positioned within the vessel to being imaging. In another example, certain components of the imaging catheter assembly 200 can be fully assembled when enclosed within the packaging container 245, while one or more other components are in an unassembled state (either enclosed within the packaging container 245 or separate from the packaging container 245).

FIGS. 4A and 4B illustrate the imaging catheter assembly 200 at distal portion 215 of the housing 205. FIG. 4A shows a perspective view of the distal portion 215 while FIG. 4B shows a cutaway perspective view of the distal portion 215.

In the exemplary embodiment shown, the distal portion 215 of the housing 205 can be configured to receive a guide wire (not shown) when the imaging catheter assembly 200 is being used to generate image data. To facilitate this, the housing 205 can include a guide wire receiving component 255 extending out from the housing 205 at the distal portion 215. The guide wire receiving component 255 may define a slot through which the guide wire passes as the distal portion 215 of the housing 205 is traversed through a vessel. In the illustrated example, this slot is defined by the guide wire receiving component 255 in combination with a portion of the housing 205 at the location along the housing 205 where the guide wire receiving component 255 extends outward from the housing 205. In some examples, the housing 205 can further include a guide wire sleeve 250 to facilitate, along with the guide wire receiving component 255, reception of the guide wire. The guide wire sleeve 250 can be coupled to the guide wire receiving component 255 and extend out distally from the guide wire receiving component 255 to define a continuous structure for receiving the guide wire. In use, a guide wire can be received in the slot of the guide wire receiving component 255 (and, when present, within the sleeve 250) and serve to guide the distal portion 215 of the housing 205 to a region of interest within the vessel of the patient to generate image data thereat.

Also located at the distal portion 215 of the housing 205 can be an imaging window 260. The imaging window 260 can be composed of a material that is substantially transparent to the frequency, or range of frequencies, of ultrasound energy emitted by the ultrasound transducer. In some cases, the imaging window 260 is located around a perimeter (e.g., circumference) of the housing 205 over a length of the housing 205 at the distal portion 215. As one example, the imaging window 260 can be made up of a number of segments fused together at the respective interfaces so as to be in direct contact and form a continuous, fluid impermeable segment of ultrasound energy transparent housing (e.g., without the presence of any adhesive material between the imaging window segments). Two or more (e.g., all) of these fused imaging window segments can be of differing flexural moduli so as to allow the housing 205 to effectively traverse a vessel while at the same time being controllable from the opposite proximal portion.

In a variety of embodiments, such as that shown here, the distal portion 215 of the housing 205 forms an enclosed space (e.g., a fluid enclosed space) without any openings. In the illustrated embodiment, this includes an enclosed space without any openings at a distal tip 262 such that the distal portion 215 includes an enclosed end space. As such, the distal portion 215 can lack any fluid ports, including instances where the housing 205 at the distal portion 215 is formed by a catheter sheath. In such embodiments, the enclosed end space of the distal portion 215 may be fluid impermeable around its perimeter over its length. In this way, the distal portion 215 is configured to prevent fluid, within the interior lumen 220 for instance, from discharging out of the catheter assembly 200 at the distal portion 215. At the same time, the distal portion 215 can seal the interior lumen 220 from fluid external to the housing 205. In applications where fluid delivery into a vessel before, during, and/or after image generation is desirable, a separate lumen (not shown) may be coupled to the housing 205 (e.g., at an exterior surface of the distal portion 215) to deliver fluid into the vessel.

As described previously, the interior lumen 220 is defined by the housing 205 and may extend from the proximal portion to the distal portion 215. Within the interior lumen 220 at the distal portion 215 is the intravascular imaging device 235, which in this example is an ultrasound transducer. The ultrasound transducer can be supported on a transducer housing 265, which is coupled to a drive cable 270 extending within the interior lumen 220. The drive cable 270 can be used to change the rotational position and/or the longitudinal position of the ultrasound transducer within the housing 205 during image data generation.

Also contained within the interior lumen 220 is an acoustic fluid medium. The acoustic fluid medium contained within the interior lumen 220 can surround, and thereby contact, the intravascular imaging device 235 and transducer housing 265 at the distal portion 215. Accordingly, when the imaging catheter assembly 200 is used to generate image data (e.g., after removing the assembly 200 from the packaging container), the acoustic fluid medium can provide a coupling medium through which ultrasound energy can be conveyed. For embodiments where the interior lumen 220 extends from the distal portion 215 to the proximal portion, a volume of the interior lumen 220 is defined along this length and may be substantially filled with the acoustic fluid medium.

In various examples, the acoustic fluid medium includes ethanol. In a particular such example, the acoustic fluid medium includes a combination of ethanol and sterile water (e.g., degassed ethanol and sterile water). For instance, the acoustic fluid medium can include between one and fifty percent by volume of ethanol, five and thirty percent by volume of ethanol, or ten and twenty percent by volume of ethanol. The ethanol in some cases may be a USP (United States Pharmacopeia) grade, biocompatible ethanol. Including ethanol as acoustic fluid medium can be useful since this acoustic fluid medium can be contained within the interior lumen 220, and in contact with the ultrasound transducer, for a prolonged period of time without detrimentally affecting properties of the ultrasound transducer, for instance due to corrosion. This can be beneficial where the imaging catheter assembly 200 is enclosed within the packaging container for a period of time prior to later use. At the same time, when the catheter housing is later removed from the packaging for use, this type of acoustic fluid medium can serve to transmit ultrasound energy between the ultrasound transducer and the surrounding vessel in a manner that provides quality image data.

Having described exemplary details related to embodiments of the imaging catheter assembly, this disclosure will now describe embodiments of methods of manufacturing such an imaging catheter assembly and well as embodiments of methods of imaging using such an imaging catheter assembly.

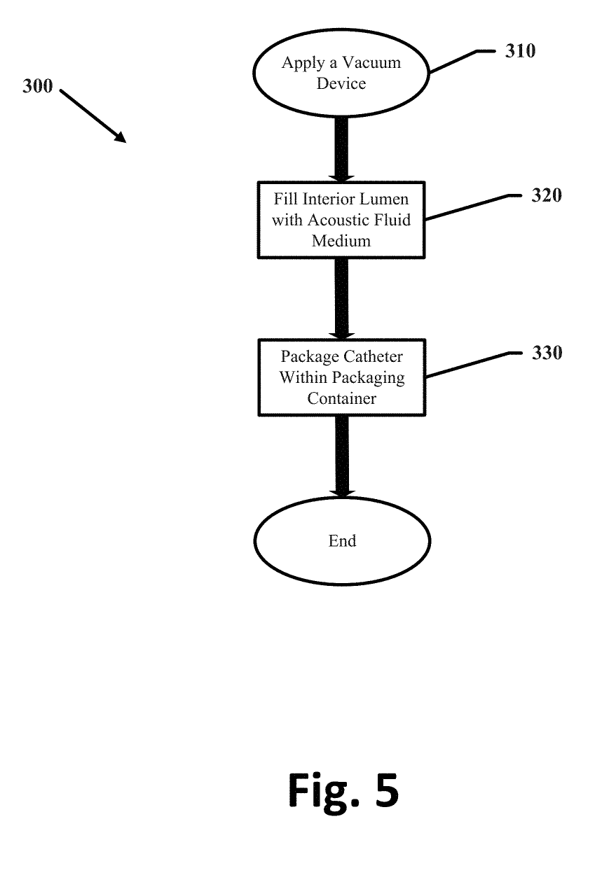

FIG. 5 shows a flow diagram illustrating an exemplary embodiment of a method 300 of manufacturing an imaging catheter assembly. The manufactured imaging catheter assembly can include any or more features disclosed herein. At step 310, a vacuum device is applied to the interior lumen defined by the catheter housing. This can remove fluid (e.g., air) from within the interior lumen. The interior lumen to which the vacuum device is applied can include an ultrasound transducer therein, such as at a distal portion thereof. In some embodiments, the vacuum device may be any device capable of inducing a negative pressure within the interior lumen of the catheter housing, one example of which is an endoflator device. In certain embodiments, applying the vacuum device to the interior lumen can include attaching the vacuum device at a proximal portion of the catheter housing. For instance, the vacuum device may be secured to a port defined at a hub portion of the catheter housing at the proximal portion, such as by engaging a first coupling component on the vacuum device with a corresponding second coupling component on the port.

At step 320, the interior lumen is filled with an acoustic fluid medium. In one example, the interior lumen can be filled with the acoustic fluid medium after the vacuum device has been applied. In another example, the interior lumen can be filled with the acoustic fluid medium while the vacuum device is being applied to the interior lumen. This may, in some instance, include filling the interior lumen with a degassed acoustic fluid medium. In this example, the acoustic fluid medium can be introduced into, and substantially fill the interior lumen, using the vacuum device. Thus, the acoustic fluid medium can be introduced into the interior lumen at a same location at which the vacuum device is applied to the interior lumen. Applying the vacuum device can impart capillary forces within the interior lumen which can act to draw the acoustic fluid medium from the proximal portion of the housing, where the acoustic fluid medium is introduced, to the distal portion of the housing, where the ultrasound transducer can be located. This can result in the volume of the interior lumen (e.g., extending from the proximal portion of the housing to the distal portion of the housing) being substantially filled with the acoustic fluid medium, including at the distal portion of the housing.

In one case, step 320 can also include hanging the catheter assembly. The catheter assembly can be hung either while introducing the acoustic fluid medium or after introducing the acoustic fluid medium, as well as either while applying the vacuum device to the interior lumen or after applying the vacuum device to the interior lumen. Hanging the catheter assembly can include positioning the catheter assembly in a generally vertical orientation where the proximal portion is at a greater elevation than the distal portion. This may allow the capillary forces to draw the acoustic fluid medium to the distal portion.

The acoustic fluid medium filled into the interior lumen can include ethanol. As described above, in one example the acoustic fluid medium can include a combination of ethanol and sterile water at a number of proportions.

At step 330, and after step 320 (e.g., after the interior lumen has been filled with the acoustic fluid medium), the catheter housing is packaged within a packing container for later use. Packaging the catheter housing within the packaging container can include enclosing the catheter housing within the packaging container. The packaging container can seal the catheter housing from an ambient environment. In this way, the catheter housing enclosed within the packaging container can include the interior lumen containing an acoustic fluid medium and the ultrasound transducer.

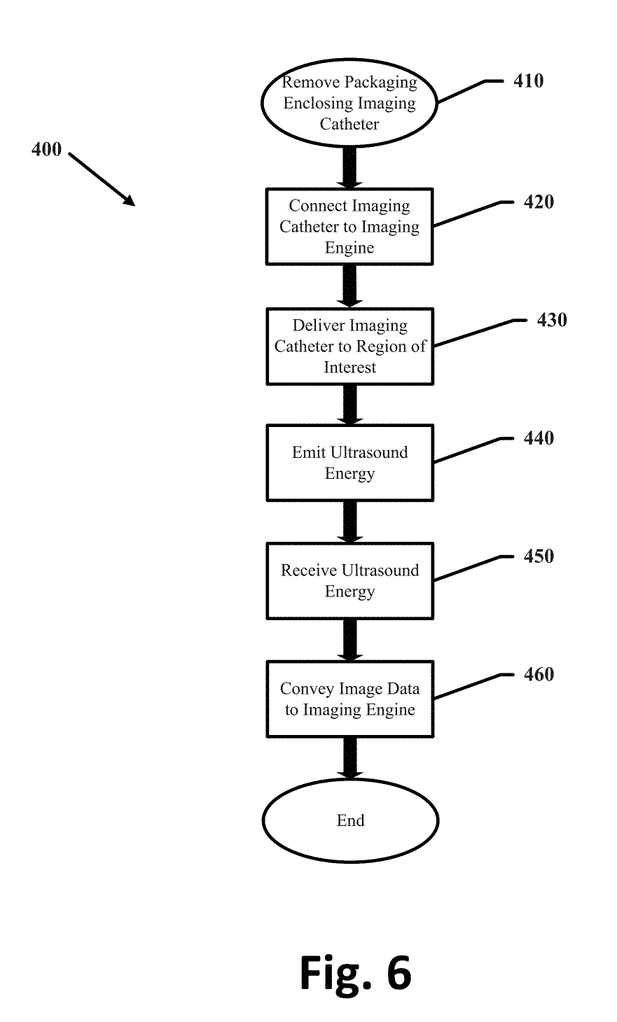

FIG. 6 shows a flow diagram illustrating an exemplary embodiment of a method 400 of imaging using an imaging catheter assembly. The imaging catheter assembly used for imaging can include any or more features disclosed herein and operate to generate and convey image data according to any of the operational details disclosed herein. At step 410, the packaging enclosing the imaging catheter is removed. The imaging catheter removed from the packaging may include a catheter housing defining an interior lumen, an ultrasound transducer disposed within the interior lumen, and an acoustic fluid medium within the interior lumen. The catheter housing may define an enclosed space without any opening at the distal portion of the catheter housing. The distal portion of the catheter housing may be fluid impermeable. As described previously, the acoustic fluid medium can include ethanol.

At step 420, and after step 410 (e.g., after removing the packaging enclosing the imaging catheter), the imaging catheter is connected (e.g., electrically) to an imaging engine. The connection between the imaging catheter and the imaging engine can facilitate two way communication therebetween. For example, generated image data can be sent from the ultrasound transducer to the imaging engine for processing and display and control signals can be sent from the imaging engine to the imaging catheter assembly. In one embodiment, there is no step involving fluid flushing of the interior lumen of the catheter housing between step 410 and step 420 (e.g., no step of fluid flushing after removing the packaging enclosing the imaging catheter and prior to connecting the imaging catheter to the imaging engine).

At step 430, and after step 410 (e.g., after removing the packaging enclosing the imaging catheter), the imaging catheter is delivered to a region of interest within a vessel of a patient. In one example, the imaging catheter can be delivered to the region of interest over a guide wire. In such an example, step 430 can include threading a guide wire through a guide wire receiving component extending out from a distal portion of the catheter housing. In this way, the guide wire can serve to facilitate the imaging catheter traversal of the vessel to the region of interest.

At step 440, ultrasound energy is emitted from the ultrasound transducer of the imaging catheter at the region of interest. Emitting ultrasound energy can include emitting ultrasound energy from the ultrasound transducer, through the acoustic fluid medium surrounding the ultrasound transducer within the interior lumen, and then into the vessel at the region of interest. Additionally, emitting ultrasound energy can include emitting the ultrasound energy through an imaging window of the catheter housing after the ultrasound energy is emitted through the acoustic fluid medium surrounding the ultrasound transducer within the interior lumen. The ultrasound energy can be emitted from the ultrasound transducer at a frequency suitable for the imaging application, for instance 40 MHz or 60 MHz. In some cases, the ultrasound energy can be emitted from the ultrasound transducer at more than one frequency.

At step 450, ultrasound energy is received at the ultrasound transducer of the imaging catheter at the region of interest. Receiving ultrasound energy can include receiving ultrasound energy reflected from the region of interest (e.g., a component within the vessel, such as tissue of non-tissue accumulations), passing through the acoustic fluid medium within the interior lumen, and then passing to the ultrasound transducer. Additionally, receiving ultrasound energy can include receiving the ultrasound energy through an imaging window of the catheter housing after the ultrasound energy is reflected from the region of interest.

At step 460, image data is conveyed from the ultrasound transducer to the imaging engine. This conveyed image data may correspond to the reflected ultrasound energy received at the ultrasound transducer at step 450. The image data can be filtered or otherwise processed at the imaging engine and displayed at the imaging engine (e.g., in substantially real-time).

In some embodiments, the method of imaging can exclude certain steps relating to preparation of the imaging catheter. For example, this method could include the steps of connecting the imaging catheter after removing the packaging, delivering the imaging catheter after removing the packaging, and emitting and receiving the ultrasound energy without introducing a flushing medium into the interior lumen of the catheter housing during these steps. Since the imaging catheter removed from the packing is pre-filled with the acoustic fluid medium, many traditional catheter preparation steps can be eliminated. This may save time, reduce user burden, and eliminate accessories previously needed to prepare an imaging catheter. Furthermore, the imaging catheter could be pre-filled using one or more techniques that reduce a likelihood that air bubbles will be entrained within the interior lumen, and consequently the pre-filled imaging catheter may reduce instances of degraded image quality.

Various examples have been described. These and other examples are within the scope of the following claims.

1. A method of manufacturing an imaging catheter, the method comprising the steps of:
applying a vacuum device to an interior lumen, the interior lumen defined by a catheter housing having an ultrasound transducer disposed within the interior lumen;
filling the interior lumen with an acoustic fluid medium, the acoustic fluid medium comprising ethanol; and
packaging the catheter housing within a packaging container for later use after filling the interior lumen with the acoustic fluid medium.

2. The method of claim 1, wherein the catheter housing packaged within the packaging container is filled with the acoustic fluid medium such that the acoustic fluid medium surrounds the ultrasound transducer disposed within the interior lumen of the catheter housing.

3. The method of claim 1, wherein the catheter housing defines a proximal portion and a distal portion and further includes a guide wire receiving component extending out from the distal portion, and wherein the distal portion forms an enclosed end space.

4. The method of claim 3, wherein the distal portion includes a distal tip that is fluid impermeable.

5. The method of claim 4, wherein the distal portion further includes an imaging window that is substantially transparent to ultrasound energy.

6. The method of claim 3, wherein applying the vacuum device to the interior lumen comprises attaching the vacuum device at the proximal portion of the catheter housing.

7. The method of claim 6, wherein attaching the vacuum device at the proximal portion of the catheter housing comprises securing the vacuum device at a port defined at a hub portion of the catheter housing.

8. A method of imaging, the method comprising the steps of:
removing packaging enclosing an imaging catheter that includes a catheter housing defining an interior lumen, an ultrasound transducer disposed within the interior lumen, and an acoustic fluid medium within the interior lumen, the acoustic fluid medium comprising ethanol;
after removing the packaging, connecting the imaging catheter to an imaging engine;
after removing the packaging, delivering the imaging catheter over a guide wire to a region of interest within a vessel;
emitting ultrasound energy from the ultrasound transducer, through the acoustic fluid medium within the interior lumen, and into the vessel at the region of interest;
receiving at the ultrasound transducer ultrasound energy reflected from the region of interest and passed through the acoustic fluid medium within the interior lumen; and
conveying image data from the ultrasound transducer to the imaging engine, wherein the image data corresponds to the reflected ultrasound energy received at the ultrasound transducer.

9. The method of claim 8, wherein the steps of connecting the imaging catheter after removing the packaging, delivering the imaging catheter after removing the packaging, and emitting and receiving the ultrasound energy are performed without introducing a flushing medium into the interior lumen of the catheter housing.

10. The method of claim 8, wherein the imaging catheter removed from the packaging defines a proximal portion of the catheter housing and a distal portion of the catheter housing, wherein the imaging catheter removed from the packaging further includes a guide wire receiving component extending out from the distal portion, and wherein the distal portion forms an enclosed end space.

11. The method of claim 10, wherein the interior lumen defined by the catheter housing of the imaging catheter removed from the packaging extends from the proximal portion to the distal portion, and wherein a volume of the interior lumen is filled with the acoustic fluid medium.

12. The method of claim 8, wherein the interior lumen defined by the catheter housing of the imaging catheter removed from the packaging is sealed from external fluid.

13. An imaging catheter assembly comprising:
a housing enclosed within a package and defining an interior lumen of the imaging catheter assembly;
an ultrasound transducer disposed within the interior lumen; and
an acoustic fluid medium contained within the interior lumen, wherein the acoustic fluid medium comprises ethanol.

14. The imaging catheter assembly of claim 13, wherein the housing defines a proximal portion and a distal portion and further includes a guide wire receiving component extending out from the distal portion, and wherein the distal portion forms an enclosed end space.

15. The imaging catheter assembly of claim 14, wherein the distal portion includes a distal tip that is fluid impermeable.

16. The imaging catheter assembly of claim 15, wherein the distal portion includes an imaging window that is substantially transparent to ultrasound energy.

17. The imaging catheter assembly of claim 14, wherein the interior lumen extends from the proximal portion to the distal portion and a volume of the interior lumen is filled with the acoustic fluid medium.

18. The imaging catheter assembly of claim 17, wherein the distal portion includes the ultrasound transducer.

19. The imaging catheter assembly of claim 18, wherein the proximal portion includes a hub portion that defines a port adapted to receive a vacuum device.

20. The imaging catheter assembly of claim 13, wherein the acoustic fluid medium contained within the interior lumen of the housing enclosed within the package comprises between ten and twenty percent by volume of ethanol.
